(19) United States
(12) Patent Application Publication
(10) Pub. No.: US 2019/0295507 A1
(43) Pub. Date: Sep. 26, 2019

(54) Adaptive Rendering of Virtual and Augmented Displays to Improve Display Quality for Users Having Different Visual Abilities

(71) Applicant: INTERNATIONAL BUSINESS MACHINES CORPORATION, Armonk, NY (US)

(72) Inventors: Tamer E. Abuelsaad, Armonk, NY (US); Ravi Tejwani, Cambridge, MA (US); Patrick Watson, Ossining, NY (US); Aldis Sipolins, New York, NY (US)

(21) Appl. No.: 15/927,776

(22) Filed: Mar. 21, 2018

(51) Int. Cl. G09G 5/37; G06T 5/00; A61B 3/11

(52) U.S. Cl. CPC G09G 5/37; G06T 5/003; G06T 5/006; G09G 2354/00; G09G 2340/0407; G09G 2340/0464; A61B 3/111

(57) ABSTRACT

A method and system of providing a synthetic reality based on visual abilities of a user are provided. Results of an eye exam of a user are determined. An individualized vision profile is created based on the determined results of the eye exam. A movement of one or more eyes of the user is tracked. For each of the one or more displays, an image is rendered on a display of the HMD by correcting graphical characteristics of the display based on the individualized vision profile and the tracked movement of the one or more eyes.

### BACKGROUND

### Technical Field

The present disclosure generally relates to virtual, augmented, and mixed reality displays, and more particularly, to improving display quality for users having different visual abilities.

### Description of the Related Art

In recent years, there has been an increase in the use of simulated environments. Virtual reality (VR) is computer-generated simulation of a stereoscopic image or environment that can be interacted with via a VR headset. For example, the VR headset provides the illusion of depth and being immersed in a scene. In contrast, augmented reality (AR) overlays virtual objects on the real-world environment. Mixed reality (MR) not only overlays virtual objects, but also anchors virtual objects in the real-world environment. For example, virtual objects are not simply overlaid on the real world but such objects can also interact with the environment. Headsets that accommodate VR, AR, and/or MR are collectively referred to herein as head-mounted displays (HMDs).

Although there are HMDs that can be adjusted for a user, they typically are not sophisticated enough to accommodate different vision problems that the eyes of a user may have. Accordingly, users typically wear additional lenses, such as contacts or glasses, in order to address at least some of their visual disorders in the context of being able to use an HMD effectively. Using additional lenses that are not integrated into the HMD can be uncomfortable and sometimes not possible due to the shape of the HMD. Further, creating a form factor for an HMD to accommodate various glasses may result in an HMD that is more bulky, costly, and less effective in providing an optimal experience to a user wearing the HMD.

### SUMMARY

According to various embodiments, a computing device, a non-transitory computer readable storage medium, and a method are provided to create a synthetic reality based on visual abilities of a user. Results of an eye exam of a user are determined. An individualized vision profile is created based on the determined results of the eye exam. A movement of one or more eyes of the user is tracked. For each of the one or more displays, an image is rendered on a display of the HMD by correcting graphical characteristics of the display based on the individualized vision profile and the tracked movement of the one or more eyes. By virtue of the adaptively rendered image, a user can enjoy a synthetic reality is based on the user's visual abilities identified in the eye exam.

In one embodiment, determining the results of the eye exam includes receiving the results of an eye exam performed separate from the HMD, via a user interface of the HMD. In other embodiments, the eye exam is performed by the HMD. In this way, the visual ability of a user can be time efficiently determined.

In one embodiment, performing the eye exam includes determining an inter-pupillary distance (IPD) of the user or determining, for each eye of the user, a focal length (FL) between the eye and a corresponding display of the HMD. Consequently, the user is provided a more comfortable visual experience based on their physical visual characteristics.

In one embodiment, the IPD or the FL are adjusted mechanically. The adjustment can be performed automatically by the HMD via one or more actuators.

In one embodiment, the IPD is adjusted by electronically shifting the image to different regions of one or more displays of the HMD, while the one or more displays are fixed with respect to the HMD.

In one embodiment, the rendered image includes a software filter correction for a distorted visual field condition identified from the results of the eye exam.

In one embodiment, the graphical characteristics of the display are corrected by, for each eye, realigning the image based on a direction of a gaze of the user or an oscillation of the eye. The graphical characteristics of the display can also be corrected by projecting visual information from a blind spot identified from the results of the eye exam and projecting the visual information from the blind spot to a functional area of a retina of the user.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all the components or steps that are illustrated. When the same numeral appears in different drawings, it refers to the same or like components or steps.

FIG. 1 illustrates an example architecture for providing a synthetic reality based on the visual abilities of a user.

FIG. 2 is a block diagram showing various components of an illustrative head mounted device at a high level, consistent with an exemplary embodiment.

FIG. 3 illustrates a perspective view of a head mounted device that is configured to adjust the inter-pupillary distance, consistent with an exemplary embodiment.

FIGS. 4A and 4B illustrate a perspective view and a zoom view, respectively, of a head mounted device that is configured to adjust a focal length between a display and a user's eyes, consistent with an exemplary embodiment.

FIG. 5 illustrates distortion correction by way of software correction, consistent with an exemplary embodiment.

FIG. 6 illustrates a foveated rendering of different regions of a display based on the tracked eye movement, consistent with an illustrative embodiment.

FIG. 7 presents a process for the adaptive rendering of a displays of an HMD based on the visual ability of a user, consistent with an illustrative embodiment.

FIG. 8 provides a functional block diagram illustration of a computer hardware platform that is capable of providing a synthetic reality.

### DETAILED DESCRIPTION

### Overview

In the following detailed description, numerous specific details are set forth by way of examples to provide a thorough understanding of the relevant teachings. However, it should be apparent that the present teachings may be practiced without such details. In other instances, well-known methods, procedures, components, and/or circuitry have been described at a relatively high-level, without detail, to avoid unnecessarily obscuring aspects of the present teachings.

The present disclosure relates to VR, AR, and/or MR, collectively referred to herein as synthetic reality. Although there are HMDs that can be adjusted for some parameters for different users, HMD's typically are not sophisticated enough to take into account various visual disorders that different users' eyes may have. To accommodate some of the visual disorders, HMDs may be configured for a user to wear their glasses (or contacts). Alternatively, a user can buy special lenses that are customized for their HMD. However, a customized HMD, or one that is manufactured specifically for a user, is typically time consuming and not cost effective. To that end, a method and system of providing a synthetic reality based on visual abilities of a user are provided. Results of an eye exam of a user are determined. An individualized vision profile is created based on the determined results of the eye exam. A movement of one or more eyes of the user is tracked. For each of the one or more displays, an image is rendered on a display of the HMD by correcting graphical characteristics of the display based on the individualized vision profile and the tracked movement of the one or more eyes.

By virtue of the concepts discussed herein, a user can enjoy a synthetic reality that that is adaptively rendered based on the user's visual abilities. Reference now is made in detail to the examples illustrated in the accompanying drawings and discussed below.

### Example Architecture

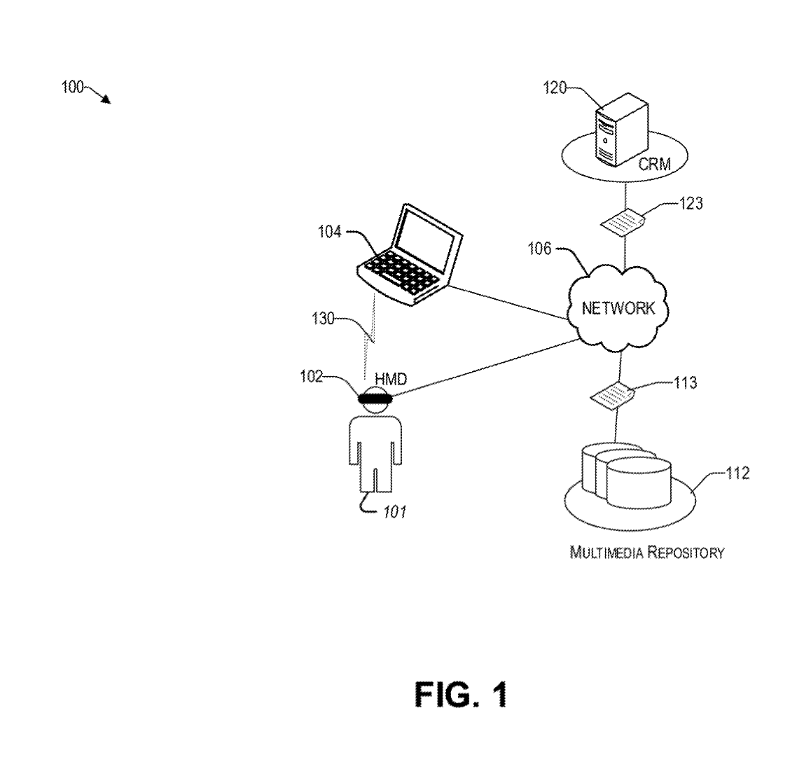

FIG. 1 illustrates an example architecture 100 for providing a synthetic reality based on the visual abilities of a user 101. There is an HMD 102 that is worn on the head or as part of a helmet of a user 101. The HMD 102 may have a display in front of one or more eyes of the user 101. In various embodiments, the HMD 102 may have a separate display for one or more eyes, or a single display to accommodate both eyes concurrently (e.g., half the display, sometimes referred to herein as a screen, is allocated for the left eye and the other half is allocated for the right eye). Different types of displays include, without limitation, TFT-LCD, IPS-LCD, OLED, AMOLED, Super AMOLED, Retina Display, etc. In one embodiment, in addition to visual feedback, the HMD 102 discussed herein may also provide additional sensory feedback, such as sound, haptic, smell, heat, and moisture.

The HMD 102 is configured to provide a virtual, augmented, and/or mixed reality experience that takes into consideration the visual disorders of the user 101 who is presently wearing the HMD 102. The HMDs discussed herein may be used in various application, such as gaming, engineering, medicine, aviation, and in scenarios where a visual acuity is to be corrected to interact with a regular environment.

In one embodiment, the architecture 100 includes a network 106 that allows the HMD 102 to communicate with other user devices, that may be in the form of portable handsets, smart-phones, tablet computers, personal digital assistants (PDAs), smart watches, business electronic devices, and other HMDs, represented by way of example in FIG. 1 as a computing device 104. The HMD 102 may also communicate with other devices that are coupled to the network 106, such as a multimedia repository 112, and a customer relationship manager (CRM 120).

The network 106 may be, without limitation, a local area network (“LAN”), a virtual private network (“VPN”), a cellular network, a public switched telephone network (PTSN), the Internet, or a combination thereof. For example, the network 106 may include a mobile network that is communicatively coupled to a private network that provides various ancillary services, such as communication with various application stores, libraries, multimedia repositories (e.g., 112), and the Internet. To facilitate the present discussion, network 106 will be described, by way of example only and not by way of limitation, as a mobile network as may be operated by a carrier or service provider to provide a wide range of mobile communication services and supplemental services or features to its subscriber customers and associated mobile device users.

As mentioned above, there may be a multimedia repository 112 that is configured to provide multimedia content 113 to the HMD 102 of a subscribed user 101. In one example, there may be a CRM server 120 that is coupled for communication via the network 106. The CRM server 120 may offer its account holders (e.g., user 101 of the HMD 102) on-line access to a variety of functions related to the user's account, such as medical information (e.g., results of an eye exam) 123, on-line payment information, subscription changes, password control, etc.

In one embodiment, a terminal, such as a computing device 104, may be used to access on-line information about a user's account, which the mobile carrier makes available via the carrier's web site accessible through the Internet. In some embodiments, the HMD may communicate with the computing device 104 to receive content therefrom via the network 106 or through short range wireless communication 130, such as Bluetooth.

While the computing device 120, CRM 110, and multimedia repository 112 are illustrated by way of example to be on different platforms, it will be understood that in various embodiments, they may be combined in various combinations, including being integrated in the HMD 102 itself. In other embodiments, the computing platforms 102 and 112 may be implemented by virtual computing devices in the form of virtual machines or software containers that are hosted in a cloud, thereby providing an elastic architecture for processing and storage.

### Example User Device

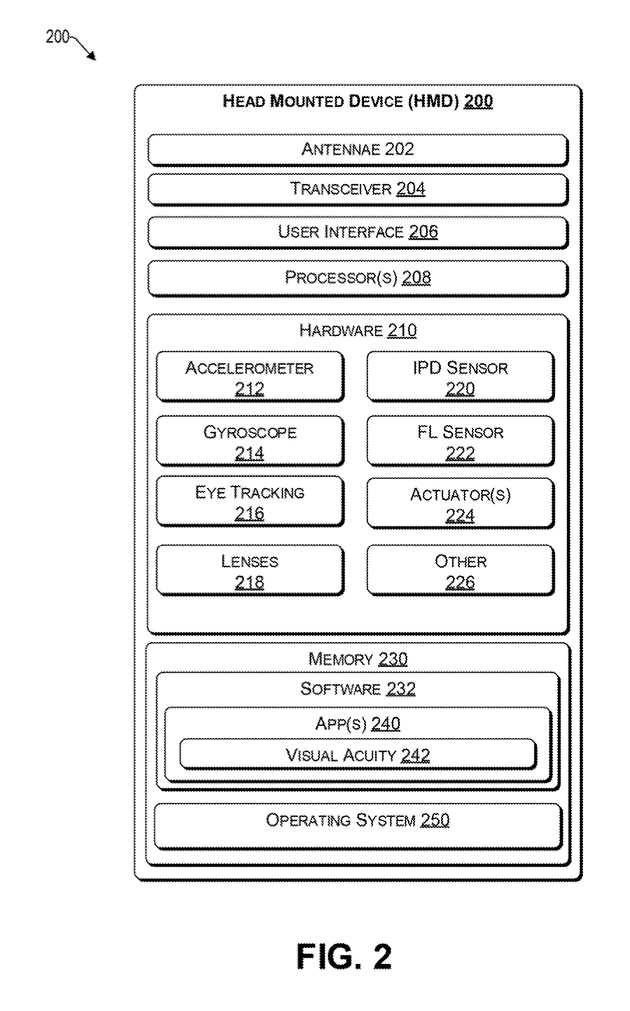

As discussed in the context of FIG. 1, the adaptive rendering of images on a display of an HMD based on the visual ability of the user may involve different types of head mounted devices. To that end, FIG. 2 illustrates a block diagram showing various components of an illustrative HMD 200 at a high level. For discussion purposes, the illustration shows the HMD 200 in the form of a wireless computing device, while it will be understood that other computing devices are contemplated as well.

The HMD 200 may include one or more antennae 202; a transceiver 204 for cellular, Wi-Fi communication, short-range communication technology, and/or wired communication; a user interface 206; one or more processors 208; hardware 210; and memory 230. In some embodiments, the antennae 202 may include an uplink antenna that sends radio signals to a base station, and a downlink antenna that receives radio signals from the base station. In some other embodiments, a single antenna may both send and receive radio signals. The same or other antennas may be used for Wi-Fi communication. These signals may be processed by the transceiver 204, sometimes collectively referred to as a network interface, which is configured to receive and transmit digital data. In one embodiment, the HMD 200 does not include an antenna 202 and communication with external components is via wired communication.

In one embodiment, the HMD 200 includes one or more user interface(s) 206 that enables a user to provide input and receive output from the HMD 200. For example, the user interface 206 may include a data output device (e.g., visual display(s), audio speakers, haptic device, etc.,) that may be used to provide a virtual, augmented or mixed reality experience to the user wearing the HMD 200.

The user interface(s) 206 may also include one or more data input devices. The data input devices may include, but are not limited to, combinations of one or more of keypads, knobs/controls, keyboards, touch screens, microphones, speech recognition packages, and any other suitable devices or other electronic/software selection interfaces. For example, the data input devices may be used by a user to enter results of an eye exam, enter and/or adjust a setting (e.g., via a knob, switch, microphone, or other electronic interface) based on a suggestion by the HMD via a user interface. 206.

The HMD 200 may include one or more processors 208, which may be a single-core processor, a multi-core processor, a complex instruction set computing (CISC) processor, gaming processor, or any other type of suitable processor.

The hardware 210 may include a power source and digital signal processors (DSPs), which may include single-core or multiple-core processors. The hardware 210 may also include network processors that manage high-speed communication interfaces, including communication interfaces that interact with peripheral components. The network processors and the peripheral components may be linked by switching fabric. The hardware 210 may include hardware decoders and encoders, a network interface controller, and/or a USB controller.

The hardware 210 may include various sensors to determine the visual ability of a user wearing the HMD 200 and/or to provide a synthetic environment to a user that accommodates their visual ability. For example, there may be one or more accelerometers 212 that are configured to measure acceleration forces, which may be used to determine an orientation of the HMD 200. There may be a gyroscope 214, which allows the measure of the rotation of the HMD, as well as lateral movements.

The hardware 210 may further include an eye tracking device 216 (e.g., a camera) to measure a position of the pupil with respect to the display (e.g., screen) in front of it. In this way, the display and/or image can be adjusted to accommodate the drift of the corresponding eye.

The hardware 210 may include one or more lenses 218 that are operative to correct one or more refractive errors of the eyes of the user. Such refractive errors that may be accommodated by the HMD 220 include myopia (i.e., nearsightedness), hyperopia (i.e., farsightedness), and astigmatism (i.e., asymmetric steepening of the cornea or natural lens that causes light to be focused unevenly). To that end, in one embodiment the lenses may be mechanically moved back and forth in front of the screen to adjust the focus based on the determined refractive error of the user. In other embodiments, one or more malleable lenses (e.g., liquid lenses) can be used to change the focus while maintaining the lenses in the same position.

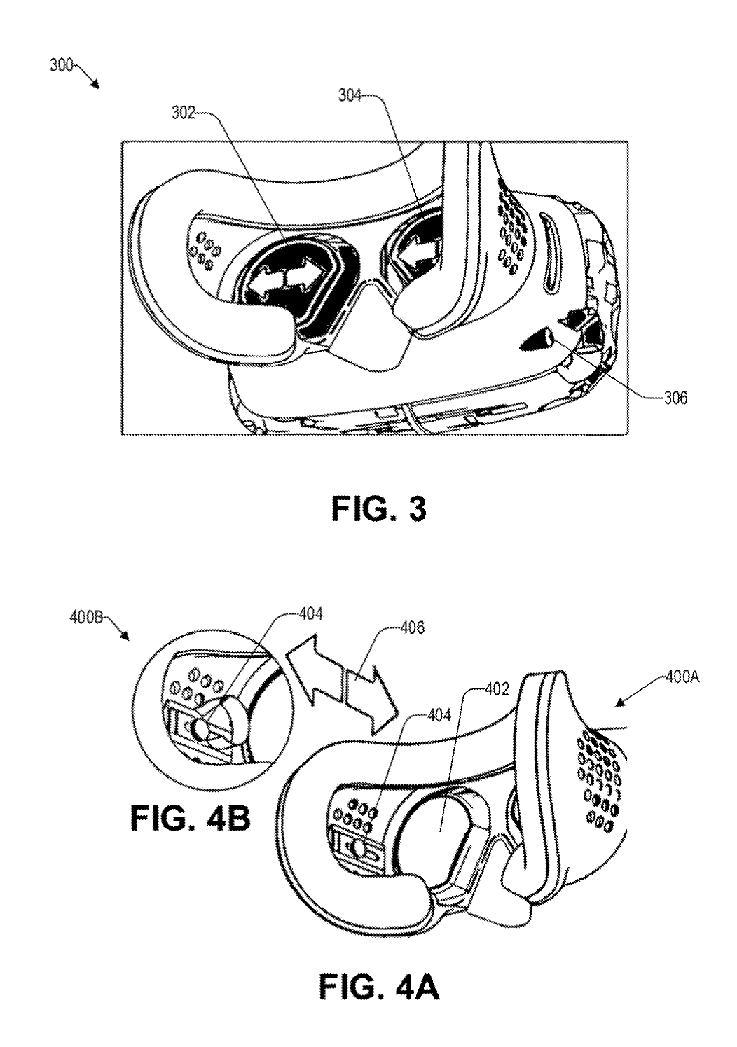

The hardware 210 may further include a sensor for inter-pupillary distance (IPD) 220. For example, there may be one or more cameras in the HMD directed towards the eyes of the user that are configured to measure the IPD. In one embodiment, the same camera used for the eye tracking can be used for the IPD measurement. The IPD adjustment is discussed in more detail later in the context of FIG. 3.

In one embodiment, the hardware 210 may include a focal length (FL) sensor 222 to determine a present distance between the display and a user's eyes. An appropriate focal length is then calculated based on the identified prescription for the user. For example, the lens maker's equation, provided below as equation 1, can be used to calculate the focal length.

$$\frac{1}{f} = ( n - 1 ) ( \frac{1}{{R}_{1}} - \frac{1}{{R}_{2}} ) \qquad ( Eq . 1 )$$

Where:
f=focal length (eye to target as computed in the virtual space);
n=index of refraction (provided by a lighting model);
R₁=the real lens radius; and
R₂=the radius of a barrel distortion applied to the image to create a virtual lensing effect.

The lens maker's equation above is a formula that provides a relationship between the focal length f, refractive index n, and radii of curvature of the two spheres used in a lens of the HMD, for relatively thin lenses (e.g., where the thickness is negligible compared to the radius of curvature). The lighting model refers to a software engine that renders the lighting in the display. For example, the lighting model includes the location of the user camera, the angle, distance to objects in the environment, and the illumination of the objects. The virtual lensing effect refers to distortion that is applied to the 3D model for the software environment to move objects, move the user camera, or bend the visual field. Unlike traditional approaches that rely on lenses to achieve these effects, the visual acuity engine can achieve these effects in virtual space based on the concepts discussed herein.

In scenarios where the thickness of the lens is not negligible with respect to the radius of the curvature of the lens, equation 2 below can be used.

$$\frac{1}{f} = ( n - 1 ) [ \frac{1}{{R}_{1}} - \frac{1}{{R}_{2}} + \frac{( n - 1 ) d}{n \times {R}_{1} \times {R}_{2}} ] \qquad ( Eq . 2 )$$

Where:
d=thickness of the subject lens.

The focal length adjustment is discussed in more detail later in the context of FIGS. 4A and 4B.

The hardware 210 may include one or more actuators 224 that are configured to automatically move a display closer to or further away from an eye of the user (i.e., adjust the focal length). There may be actuators 224 that automatically change the IPD between two displays. Other actuators may perform other automatic functions.

The hardware 210 may also include other sensors 226 that may operate in addition to or instead of the above-mentioned sensors to determine the cylindrical lens correction, the lens meridian (e.g., Axis), the added magnifying power (e.g., Add), the prismatic power (e.g., prism), diopter magnification, visual field direction, pupillary dilation, and eye rotation of the user.

The HMD 200 includes memory 230 that may be implemented using computer-readable media, such as computer storage media. Storage media includes volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD), high definition video storage disks, or other optical storage, magnetic cassettes, magnetic tape, magnetic disk storage or other magnetic storage devices, or any other non-transmission medium that can be used to store information for access by a computing device.

The memory 230 may store various software components or modules that are executable or accessible by the processor(s) 208 and controller(s) of the HMD 200. The various components of the memory 230 may include software 232 and an operating system 250. The software 232 may include various applications 240, such as a visual acuity engine 242 having several modules, each configured to control a different aspect the determination of the visual ability of a user and the rendering of images on the display of the HMD 200 based on the visual ability of the user. Each module may include routines, program instructions, objects, and/or data structures that perform tasks or implement abstract data types, discussed in more detail later.

The operating system 250 may include components that enable the HMD 200 to receive and transmit data via various interfaces (e.g., user controls, communication interface, and/or memory input/output devices), as well as process data using the processor(s) 208 to generate output. The operating system 250 may include a presentation component that presents the output (e.g., display the data on an electronic display of the HMD 200, store the data in memory 230, transmit the data to another electronic device, etc.). Additionally, the operating system 250 may include other components that perform various additional functions generally associated with an operating system 250. By virtue of the hardware and software of the HMD 200, a user can enjoy an elevated visual experience that is tailored to their visual ability, whether with or without glasses or contacts.

### Example Visual Corrections of the Head Mounted Device

Reference now is made to FIG. 3, which illustrates a perspective view of an HMD 300 that is configured to adjust the IPD, consistent with an exemplary embodiment. For example, the IPD may be determined by HMD for each eye of the user. In various embodiments, the IPD may be received as an input to the HMD 300 or automatically determined by the one or more sensors of the HMD 300, as discussed previously. The IPD can then be adjusted between the center of the pupils of the two eyes by moving the left display 302 and right display 304 accordingly. Alternatively, the image may be shifted electronically (to different regions of each display) instead of mechanical adjustment of the displays. Stated differently, while the display remains fixed, the image thereon is shifted through software to accommodate the determined IPD of the particular user.

In one embodiment, the IPD adjustment is performed automatically by one or more actuators of the HMD 200 that are configured to move the displays 302 and 304 with respect to the determined IPD setting of the user. Alternatively (e.g., in HMDs that do not have such actuator(s)), the HMD instructs the user via a user interface of the HMD by providing a calculated setting via an input device, represented by way of example, and not by way of limitation, as a rotatable knob 306. For example, the HMD may indicate that a particular setting on an appropriate scale (e.g., a setting of 8 on a scale of 1-10) is the correct IPD setting for the user. The user can then dial the setting (e.g., 8) via the input device 306 to mechanically adjust the IPD.

FIGS. 4A and 4B illustrate a perspective view 400A and a zoom view 400B, respectively, of an HMD that is configured to adjust a focal length between a display 402 and a user's eyes, consistent with an exemplary embodiment. Similar to the IPD, the appropriate focal length may be received as an input by the HMD 300 or may be automatically determined by the one or more sensors of the HMD 300. The distance between the display 402 and the user's eyes can then be adjusted 406 by moving the display 402 closer or further away from the eyes automatically by the HMD by way one or more actuators or by the user. For example, the HMD may calculate an appropriate setting, which is then entered via an input device, represented by way of example, and not by way of limitation, as a rotatable knob 404.

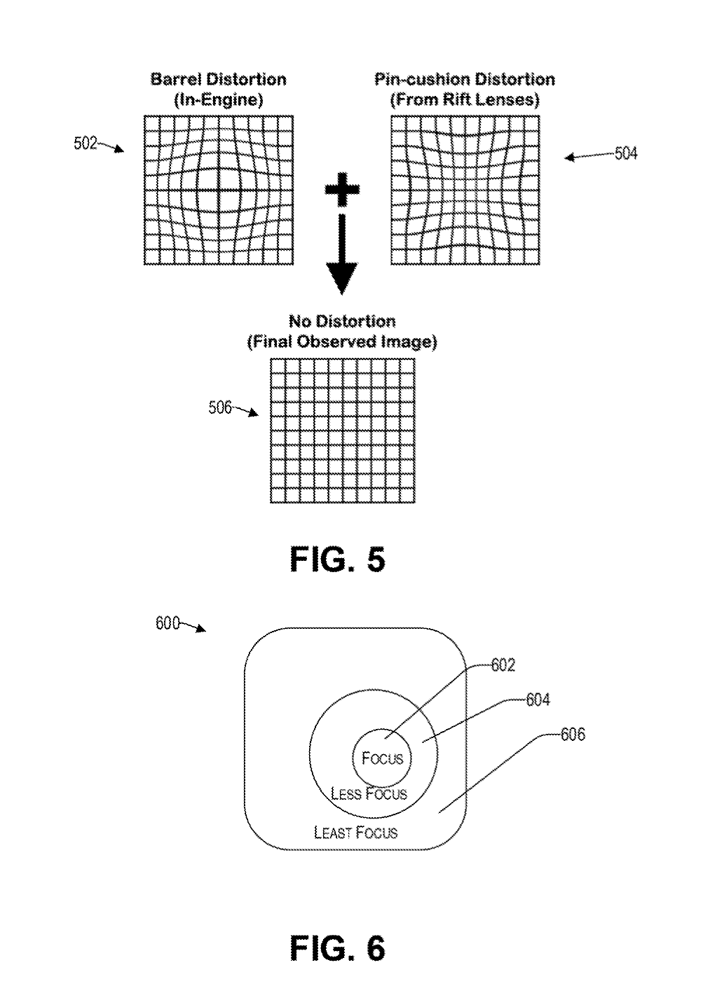

The lenses of the HMD and/or glasses or contacts worn by the user may have barrel distortion or pincushion distortion that may affect the quality of the vision of a user. Reference now is made to FIG. 5, which illustrates an example of distortion correction by way of software correction, consistent with an exemplary embodiment. For example, there may be an inherent pincushion distortion 504 due to the lenses of the HMD. Replacing the lenses with more sophisticated lenses to avoid or reduce such distortion may not be cost effective. Instead, in one embodiment, one or more sensors of the HMD may identify this distortion and correct it by way of a software filter that adds barrel distortion 502 to the rendered image such that no distortion is visible 506 to the user wearing the HMD. Stated differently, the visual acuity engine of the HMD creates an image that counteracts the effects of pincushion distortion or barrel distortion by way of a software correction of the image.

In one embodiment, the HMD uses its eye tracking module to adjust the display resolution in different regions of a display. In this regard, FIG. 6 illustrates a foveated rendering of different regions of a display 600 based on the tracked eye movement, consistent with an illustrative embodiment. Foveated rendering blurs the image based on a distance from the tracked eye focus. For example, the HMD may determine that the eye is focused to region 602. Accordingly, more processing power is allocated to the region 602 such that it is highest focus. The region next to it 604 may be in less focus, and the region further away 60 may be in least focus. By virtue of such foveated rendering by way of eye tracking, valuable processing power is conserved and the user is provided with a more responsive and dynamic image.

Disorders involving a distorted visual field can be corrected via modulation of the rendering to create an image that is clear from the user's perspective. For example, the epiretinal membrane, which is a thin sheet of fibrous tissue that sometimes develops on the surface of the macular area of the retina, may cause a disturbance in vision. This disturbance is identified by the sensors of the HMD and corrected by the visual acuity engine such that an image is rendered on the display that is perceived by the user to have no distortions.

In another example, the sensors of the HMD can identify Keratoconus, which is a disorder of the eye that results in a thinning of the cornea, which is perceived by a user as blurry vision, double vision, nearsightedness, astigmatism, and light sensitivity.

As discussed before in the context of FIG. 3, eye movement disorders can be corrected by static or dynamic shifting of the visual field to accommodate for visual defects by using eye tracking. For example, strabismus (sometimes referred to as cross eye) and double vision are corrected by tracking the eye and realigning the image based on a direction of the gaze. Similarly, Nystagmus (where the eye makes repetitive uncontrolled movements that may result in reduced vision and depth perception) and amblyopia (sometimes referred to as lazy eye) can be corrected by the HMD by tracking the eye movement and realigning the images displayed based on the oscillation of the eye. For abducens paralysis, a disorder associated with dysfunction of the cranial nerve, the image may be rotated to accommodate the eye.

In one embodiment, obscuring disorders that occlude a portion of the visual field can be improved by the visual acuity engine by distorting the visual field to re-project visual information from blind spots to functional areas of the retina.

### Example Scenarios

As mentioned previously, the HMDs discussed herein may be used in various application, such as gaming, engineering, medicine, aviation, and where a visual acuity is to be corrected. In this regard, it may be helpful to discuss some example non-limiting scenarios. In a first scenario, a first user may have a refractive error (e.g., nearsightedness), but may find it inconvenient to fit the glasses in the HMD. The first user therefore removes the glasses and takes an interactive HMD eye exam. An eye exam can involve an estimation of a map describing visual distortions, motion, occlusion, and astigmatism across the first user's visual field. The exam is administered by displaying a visual scene with spatially distributed objects. The first user is asked to identify objects (such as letters), to fixate on particular locations of the visual scene and identify peripheral objects, and to select between visual filters that they prefer. The first user may also be asked to enter other information about their visual experience.

Based on the results of the eye exam, the HMD can be adjusted to accommodate the visual ability of the first user. The adjustment can be performed (i) automatically by the visual acuity engine of the HMD by applying one or more software filters that render an image on a display of the HMD based on the visual ability of the user and/or (ii) mechanically adjust one or more parameters of the HMD, such as the IPD and focal length. In one embodiment, at least some of the adjustments (e.g., IPD and focal length) are performed by the first user based on settings calculated by the visual acuity engine. Accordingly, while the HMD determines the correct setting, the mechanical energy of the user is used to implement the setting. In this way, the HMD accommodates the refractive error of the first user.

In a second scenario, consider a second user wearing corrective contact lenses with the HMD. The HMD performs an interactive eye exam to determine the visual ability of the second user while the second user is wearing the corrective contact lenses. In this way, the HMD can identify issues that were not addressed by the contact lenses, thereby providing a better visual experience with the HMD. To that end, based on the results of the eye exam, adjustments can be performed automatically via (i) software or (ii) mechanically, to accommodate the visual ability of the second user. For example, the IPD and the focal length can be adjusted. As mentioned above, at least some of the adjustments can be performed by the second user based on settings provided by the visual acuity engine on a user interface of the HMD.

In a third scenario, a third user already has the results of an eye exam, which may be retrieved from a remote repository via a network 106, such as a CRM of FIG. 120, manually entered into the HMD via a user interface of the HMD, or scanned by the HMD via a QR or bar code provided by the third user. Accordingly, the HMD need not perform an interactive eye exam on the third user but can rely on the received results of an eye exam that was performed somewhere else. Based on the results of the eye exam, adjustments can be performed automatically by the visual acuity engine (i) via software or (ii) mechanically to accommodate the visual ability of the second user. For example, the IPD and the focal length can be adjusted. As mentioned above, at least some of the adjustments can be performed by the second user based on settings provided on a user interface of the HMD.

In a fourth scenario, consider a fourth user who has an epiretinal membrane that distorts part of his visual field. Instead of performing surgery or referring to corrective lenses, the fourth user can use the HMD as a mixed reality pass-through camera that is configured to accommodate his visual ability. In one embodiment, a calibration of the HMD can be performed (i) after every eye exam performed by the HMD or (ii) after the HMD receiving results of an eye exam conducted remotely.

### Example Process

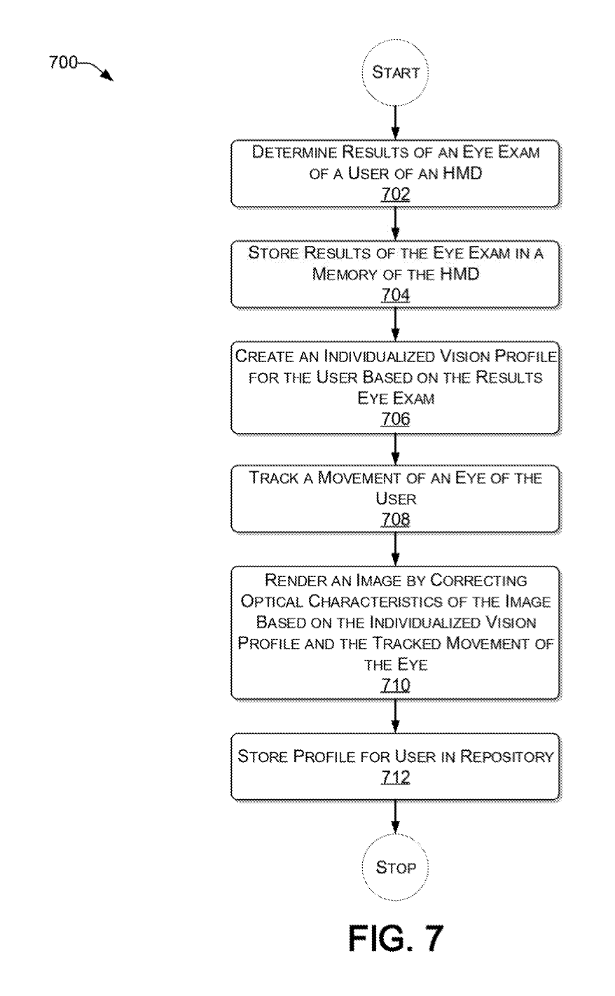

With the foregoing overview of the architecture 100, example HMD 200, and example scenarios, it may be helpful now to consider a high-level discussion of an example process in the form of a flow chart. To that end, FIG. 7 presents a process 700 for the adaptive rendering of a displays of an HMD based on the visual ability of a user, consistent with an illustrative embodiment.

Call flow 700 is illustrated as a collection of processes in a logical flowchart, which represents a sequence of operations that can be implemented in hardware, software, or a combination thereof. In the context of software, the processes represent computer-executable instructions that, when executed by one or more processors, perform the recited operations. Generally, computer-executable instructions may include routines, programs, objects, components, data structures, and the like that perform functions or implement abstract data types. The order in which the operations are described is not intended to be construed as a limitation, and any number of the described operations can be combined in any order and/or performed in parallel to implement the process. For discussion purposes, the 700 is described with reference to the architecture 100 of FIG. 1.

In process 700, a user interacts with an HMD 102 to enjoy the content provided thereby via one or more displays of the HMD 102 that provide a synthetic reality based on the visual ability of the user. At block 702, the visual acuity engine of the HMD 102 determines the results of an eye exam of the user. For example, the eye exam may be performed interactively by the HMD 102. Alternatively, or in addition, results of an eye exam that was performed somewhere else are received by the HMD 102. The eye exams performed may include, without limitation, refractive errors, IPD, FL, visual field distortions, thickness of the cornea, double vision, light sensitivity, eye movement disorders, nystagmus, etc.

In one embodiment, at block 704, the results of the eye exam are stored in a memory of the HMD 102. The corpus of the stored eye exam data can then be used by the visual acuity engine to determine a visual ability of the user.

At block 706, an individualized vision profile is created for the user by the visual acuity engine, based on the results of the eye exam in general and the determined visual ability of the user in particular. This custom profile of the user includes different software filters and/or mechanical adjustments to counteract the distortions to the user.

At block 708, the movement of one or more eyes of the user are tracked to measure a position of the pupil with respect to the display of the HMD 102. In this way, the display can later be adaptively adjusted to accommodate the drift of each eye.

At block 710, an image is rendered on the display of the HMD 102 by correcting graphical characteristics of the display based on the individualized vision profile and the tracked movement of the eye. To that end, software adjustments are performed by the acuity engine of the HMD 102 by way of one or more software filters that are applied to a rendering engine of the HMD 102 to render images that counteract the visual distortions of the user identified in the results of the eye exam.

In some embodiments, mechanical adjustments are performed in addition to the software adjustments. These adjustments can be performed automatically by the HMD 102 via one or more actuators. Alternatively, or in addition, the mechanical adjustments are performed by the user based on settings provided by the visual acuity engine. For example, the HMD instructs the user to move a mechanical input device, such as a mechanical lever or knob, to a specified position. In this way, the user need not determine an optimal setting, but merely provides the mechanical power to make an adjustment that is determined by the visual acuity engine based on the visual ability of the user.

In one embodiment, at block 712, the profile setting is stored in a suitable repository, such as a memory of the HMD 102 or the CRM 120. In this way, the upon identifying the user, the HMD 102 need not perform an eye test or retrieve results of an eye test; rather, the individualized vision profile can be loaded from the memory of the HMD 102 or the CRM 120.

### Example Computer Platform

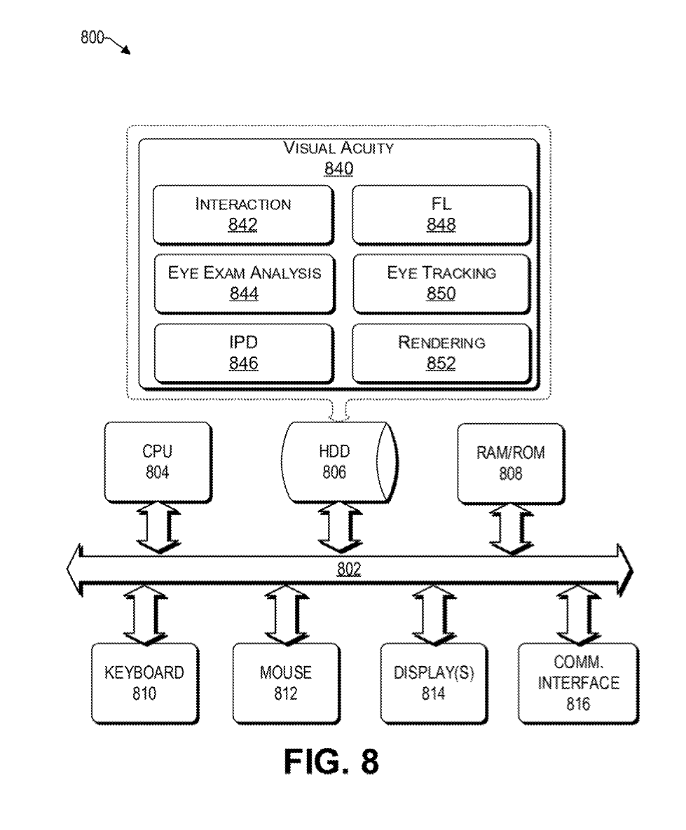

As discussed above, functions relating to providing adaptive rendering of images to create a synthetic reality based on the visual abilities of a user can be performed with the use of one or more computing devices that may be connected for data communication via wireless or wired communication, as shown in FIG. 1 and in accordance with the process 700 of FIG. 7. An example computing device in the form of an HMD 200 has been discussed above with respect to FIG. 2. FIG. 8 provides a functional block diagram illustration of a computer hardware platform that is capable of providing a synthetic reality. In particular, FIG. 8 illustrates a computer platform 800, as may be used to implement a computing device such as the HMD 102.

The computer platform 800 may include a central processing unit (CPU) 804, a hard disk drive (HDD) 806, random access memory (RAM) and/or read only memory (ROM) 808, a keyboard 810, an input device (e.g., mouse) 812, one or more displays 814, and a communication interface 816, which are connected to a system bus 802.

In one embodiment, the HDD 806, has capabilities that include storing a program that can execute various processes, such as the visual acuity engine 840, in a manner described herein. The visual acuity engine 840 may have various modules configured to perform different functions.

For example, there may be an interaction module 842 that is operative to receive results of eye tests via a user interface, such as a keyboard 810, mouse 812, touch sensitive display 814, etc., or over a network via the communication interface 816. The interaction module 842 can also provide instructions to users on a user interface, such as the calculated settings of the HMD. The interaction module 842 may also interact with a CRM to store and/or retrieve an individualized vision profile information of a user.

In one embodiment, there is an eye exam analysis module 844 operative to determine an individualized vision profile for a user based on the results of the eye exam.

In one embodiment, there is an IPD module 846 operative to cooperate with the IPD sensor 220 to determine a distance between the center of the pupils and calculate an optimal distance between two displays (e.g., left and right) of the HMD, accordingly. Alternatively, the image may be shifted electronically to different regions of each display (or single display) instead of mechanical adjustment of the displays. Stated differently, different regions of a display are used instead of mechanically moving the display.

In one embodiment, there is an FL module 848 operative to cooperate with the FL sensor 222 to determine a distance between the display and a user's eyes and calculate an optimal setting thereof using the equations discussed herein.

In one embodiment, there is an eye tracking module 850 operative to cooperate with the eye tracking sensor 216 to measure a position of the pupil with respect to the display in front of it. In one embodiment, the tracking module 850 can dynamically calculate what regions on the display merit better focus, thereby conserving processing power and providing better responsiveness to the user.

There is a rendering module 852 that is operative to render images on the display(s) 814 that accommodate the visual ability of the user based on input from various sensors discussed herein.

In one embodiment, a program, such as Apache™, can be stored for operating the system as a Web server. In one embodiment, the HDD 806 can store an executing application that includes one or more library software modules, such as those for the Java™ Runtime Environment program for realizing a JVM (Java™ virtual machine).

### CONCLUSION

The descriptions of the various embodiments of the present teachings have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the foregoing has described what are considered to be the best state and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

The components, steps, features, objects, benefits and advantages that have been discussed herein are merely illustrative. None of them, nor the discussions relating to them, are intended to limit the scope of protection. While various advantages have been discussed herein, it will be understood that not all embodiments necessarily include all advantages. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Numerous other embodiments are also contemplated. These include embodiments that have fewer, additional, and/or different components, steps, features, objects, benefits and advantages. These also include embodiments in which the components and/or steps are arranged and/or ordered differently.

Aspects of the present disclosure are described herein with reference to a flowchart illustration and/or block diagram of a method, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the FIGS. herein illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing has been described in conjunction with exemplary embodiments, it is understood that the term “exemplary” is merely meant as an example, rather than the best or optimal. Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments have more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A head mounted device (HMD) comprising:
a processor;
a storage device coupled to the processor;
one or more displays coupled to the processor;
a visual optimization software stored in the storage device, wherein an execution of the software by the processor configures the HMD to perform acts comprising:
determining results of an eye exam of a user;
creating an individualized vision profile based on the determined results of the eye exam;
tracking a movement of one or more eyes of the user; and
for each of the one or more displays, rendering an image on the display by correcting graphical characteristics of the display based on the individualized vision profile and the tracked movement of the one or more eyes.

2. The HMD of claim 1, wherein determining the results of the eye exam comprises:
receiving the results of an eye exam performed separate from the HMD, via a user interface of the HMD; and
storing the results of the eye exam in the storage device.

3. The HMD of claim 1, wherein determining the results of the eye exam comprises:
performing the eye exam on the user by the HMD; and
storing the results of the eye exam in the storage device.

4. The HMD of claim 3, wherein performing the eye exam comprises at least one of:
determining an inter-pupillary distance (IPD) of the user;
determining, for each eye of the user, a focal length (FL) between the eye and one of the one or more displays.

5. The HMD of claim 4, further comprising mechanically adjusting at least one of: (i) the IPD and (ii) the FL.

6. The HMD of claim 5, wherein the mechanical adjustment is performed automatically by the HMD via one or more actuators.

7. The HMD of claim 4, further comprising adjusting the IPD by electronically shifting the image to different regions of the one or more displays, while the one or more displays are fixed with respect to the HMD.

8. The HMD of claim 1, wherein the rendered image is three dimensional (3D).

9. The HMD of claim 1, wherein
the one or more displays are a single fixed display; and
for each eye, the rendered image is moved to different regions of the display based on a focal point of the eye determined from the tracked movement of the eye.

10. The HMD of claim 1, wherein execution of the software by the processor further configures the HMD to perform acts comprising: adjusting an adjustable lens of the HMD to accommodate a refractive error identified from the results of the eye exam.

11. The HMD of claim 1, wherein the rendered image includes a software filter correction for compensating for barrel or pincushion distortion of one or more lenses of the HMD.

12. The HMD of claim 1, wherein correcting the graphical characteristics of the display based on the individualized vision profile and the tracked movement of the one or more eyes comprises, for each eye, providing a foveated rendering based on the tracked movement of the one or more eyes.

13. The HMD of claim 1, wherein the rendered image includes a software filter correction for a distorted visual field condition identified from the results of the eye exam.

14. The HMD of claim 1, wherein correcting the graphical characteristics of the display based on the individualized vision profile and the tracked movement of the one or more eyes comprises, for each eye, realigning the image based on at least one of: (i) a direction of a gaze of the user, and (ii) an oscillation of the eye.

15. The HMD of claim 1, wherein correcting the graphical characteristics of the display based on the individualized vision profile and the tracked movement of the one or more eyes comprises, for each eye, projecting visual information from a blind spot identified from the results of the eye exam and projecting the visual information from the blind spot to a functional area of a retina of the user.

16. A non-transitory computer readable storage medium tangibly embodying a computer readable program code having computer readable instructions that, when executed, causes a head mounted device (HMD) to carry out a method of providing a synthetic reality based on visual abilities of a user, the method comprising:
determining results of an eye exam of a user;
creating an individualized vision profile based on the determined results of the eye exam;
tracking a movement of one or more eyes of the user; and
for each of the one or more displays, rendering an image on a display of the HMD by correcting graphical characteristics of the display based on the individualized vision profile and the tracked movement of the one or more eyes.

17. The non-transitory computer readable storage medium of claim 16, wherein determining the results of the eye exam comprises receiving the results of an eye exam performed separate from the HMD, via a user interface of the HMD.

18. The non-transitory computer readable storage medium of claim 16, wherein determining the results of the eye exam comprises performing the eye exam on the user by the HMD.

19. The non-transitory computer readable storage medium of claim 18, wherein performing the eye exam comprises at least one of:
determining an inter-pupillary distance (IPD) of the user;
determining, for each eye of the user, a focal length (FL) between the eye and a corresponding display of the HMD.

20. The non-transitory computer readable storage medium of claim 19, further comprising mechanically adjusting at least one of: (i) the IPD and (ii) the FL.

21. The non-transitory computer readable storage medium of claim 19, further comprising adjusting the IPD by electronically shifting the image to different regions of one or more displays of the HMD, while the one or more displays are fixed with respect to the HMD.

22. The non-transitory computer readable storage medium of claim 16, wherein the rendered image includes a software filter correction for a distorted visual field condition identified from the results of the eye exam.

23. The non-transitory computer readable storage medium of claim 16, wherein correcting the graphical characteristics of the display based on the individualized vision profile and the tracked movement of the one or more eyes comprises, for each eye, at least one of:
realigning the image based on at least one of: (i) a direction of a gaze of the user, and (ii) an oscillation of the eye; and
projecting visual information from a blind spot identified from the results of the eye exam and projecting the visual information from the blind spot to a functional area of a retina of the user.

24. A method comprising:
determining results of an eye exam of a user by a head mounted device (HMD);
creating an individualized vision profile based on the determined results of the eye exam by the HMD;
tracking a movement of one or more eyes of the user by the HMD; and
for each of the one or more displays, rendering an image on a display of the HMD by correcting graphical characteristics of the display based on the individualized vision profile and the tracked movement of the one or more eyes.

25. The method of claim 24, wherein:
determining the results of the eye exam comprises:
performing the eye exam on the user by the HMD; and
storing the results of the eye exam in the storage device; and
performing the eye exam comprises at least one of:
determining an inter-pupillary distance (IPD) of the user;
determining, for each eye of the user, a focal length (FL) between the eye and one of the one or more displays.
